(19) United States
(12) Patent Application Publication
(10) Pub. No.: US 2002/0159975 A1
(43) Pub. Date: Oct. 31, 2002

(54) DERIVATION OF CELLS AND TISSUES FROM EMBRYONIC PRE-STEM CELLS FOR TRANSPLANTATION THERAPIES

(76) Inventor: GARY D. HODGEN, VIRGINIA BEACH, VA (US)

Correspondence Address: Edward A. Meilman, Dickstein Shapiro Morin & Oshinsky LLP, 1177 Avenue of the Americas, 41st floor, New York, NY, 10036-2714, US

(21) Appl. No.: 09/327,388

(22) Filed: Jun. 7, 1999

(51) Int. Cl. A61K 48/00; A01K 67/00

(52) U.S. Cl. 424/93.1; 424/93.21; 800/13; 800/21; 435/325

(57) ABSTRACT

A novel method of isolating and propagating a line of embryonic stem cells that originates from either morulae (pre-stem) or blastocyst (ICM stem cells) is disclosed for the purpose of transplanting cells, tissues or organs.

### BACKGROUND OF THE INVENTION

The present invention relates to the derivation of cells and tissues from embryonic pre-stem cells for transplantation therapies.

### SUMMARY OF THE INVENTION

This invention relates to the use of dispersed morula cells in preference to inner cell mass (ICM) from blastocysts. The morula stage is the last pre-embryonic stage without expression of any differentiation, making these cells (pre-stem cells) all progenitors of embryonic stem cells (ESCs) later present in blastocysts. Conversely, the ICM from the blastocyst is already differentiated from trophoblastic cells, which are by then destined to become part of the placenta.

This invention also relates to the use of chimeric introductions into pre-stem cell cultures and stem cell propagations in culture. That is, “teacher-cells” or spent media from them, that derived from other sources (e.g. adults, cord blood, fetal tissues, etc.) will “teach” undifferentiated pre-stem cells how to convert to our sought-after therapeutic cell population both more rapidly and more preferentially.

This invention also relates to the identification and use of certain early markers of stem cell differentiation, such as Fe++ sequestration, hemoglobin accumulation, myeloid fibers, insulin synthesis, dopamine loading, etc.

Other features and advantages of the present invention will become apparent from the following description of the invention.

### DETAILED DESCRIPTION OF THE INVENTION

The present invention provides for a method of isolating and propagating a line of embryonic stem cells that originates from either morulae (pre-stem) or blastocyst (ICM stem cells). Therefore, Morula stage, undifferentiated pre-stem cells will be used as progenitors of stem cell populations. The propagated line of embryonic stem cells are then used for the purpose of transplanting cells, tissues or organs.

The propagation of stem cells can be initiated by formation of chimeric inner cell mass cells. Chimeric ICMs will be developed from blastocysts. From such ICMs, superior stem cell cultures are derived. Preferably, the formation of chimeric inner cell mass cells comprises nuclear transplantation, mitochondrial substitution, or cytoplasmic depletion.

Preferably, at least one regulatory factor is used to propagate the line of embryonic stem cells. More preferably, the regulatory factor is derived from “Teacher cells” or “Teacher cells'” spent culture medium. “Teacher cells” will be introduced into less differentiated pre-stem or early stage stem cells to accelerate propagation of target stem cells. Alternatively, spent media from “teacher cells” can be used to accelerate the propagation of the target stem cells.

In a preferred embodiment, the embryonic stem cells are cultured in a medium in the presence of at least one agent or cytokine in order to differentiate into target specific cells or tissues. Preferably, the agent or cytokine is selected from the group consisting of IL-1, TNF-α, IL-6, PTH, PDGF, PGE₂, cAMP, estrogens, anti-estrogens, progestins, anti-progestins, cortisol, GH, androgens, I₃/T₃, VGEF and cyclosporin. Also preferably, the concentration of the agent or cytokine in culture medium is from about 1.0 pg/ml to about 10.0 ng/ml.

In another preferred embodiment, the target specific cells are selected from the group consisting of nerve cells, bone cells, immune cells, and pancreatic beta cells.

Techniques and parameters for the use of a broad spectrum of early stage metabolic markers are developed. Some such markers are, for example: Fe++ sequestration, Hg accumulation, myeloid fibers, nerve growth factor, apoptotic factors, insulin synthesis, dopamine loading, hemoglobin loading, etc. Additionally, other early markers of embryonic stem cells can be identified.

Specific techniques are developed to demonstrate the foregoing. In one embodiment of the invention, embryonic stem (ES) cells are derived from either morula or blastocyst stage embryos by placing cells on fibroblast feeder layers. The colonies are evaluated for differentiation state using accepted markers. Further evaluation is done by injecting ES cells into surrogate embryos to produce chimeras and evaluating the contribution of the ES cells to the adult tissues. Finally, ideal colonies of ES cells are used as nuclear donors for nuclear transplantation.

Clonal properties of the propagated stem cells can be achieved by adding apoptotic factors, cytokines or other agents to the culture medium to eliminate contaminating members of the stem cells that did not properly differentiate. Preferably, the cytokines or agents are selected from the group consisting of IL-1, TNF-α, IL-6, PTH, PDGF, PGE₂, cAMP, estrogens, anti-estrogens, progestins, anti-progestins, cortisol, GH, androgens, I₃/T₃, VGEF and cyclosporin.

Alternatively, the propagation of the line of embryonic stem cells is done in vivo by transplanting “Teacher cells” into an area sufficiently close to the embryonic stem cells to allow for at least one regulatory factor made by the teacher cells to contact the embryonic cells.

The presence or absence of different concentrations of calcium can be used to regulate the propagation of the line of embryonic stem cells.

Preferably, the propagated line of embryonic stem cells is grown in a three dimensional manner before being used for transplantation.

This invention will allow for the efficient, safe and commercially viable derivation of cells and tissues from embryonic pre-stem cells for transplantation therapies. Specifically, growing-out of human blastocysts at a rate greater than 50% from the 2-cell stage of the pre-embryo should be achieved. Also, efficient harvesting of either morula stage pre-stem cells and/or stem cells isolated from the inner cell mass of blastocysts should be achieved. These embryonic pre-stem and stem cell populations should preferably remain viable in culture for more than one week.

### EXAMPLE 1

Clonal production of stem cells will be undertaken. These clones will respond to the ambient levels of glucose in their milieu, and in turn, insulin-dependent diabetes would be treated by transplanting these stem cell lines to served by a peripheral blood supply. the insulin secretory cells must accomplish renewal y propagation through mitogenic proliferation.

### EXAMPLE 2

Pluripotent stem cells will be isolated and directed to differentiate into hemopoietic destinies. Therefore, tissues derived from the blood cell group or beta cells of the immune response system will be replaced in deficient patients suffering from conditions such as HIV infection, post-chemotherapy, or irradiation depletion. Culture condition in vitro will direct the rate and degree of differentiation manifested by these pluripotent stem cells, such as the presence of “teacher cells” or certain additives to the media, e.g. cytokines.

### EXAMPLE 3

The inherent capabilities of stem cells will be modified by formation of chimeric cell lines that incorporate “hybrid” metabolic functions that when transplanted will provide the transplant recipient with long-term relief from organ/tissue deficiencies. For instance, the production of dopamine in situ can modify neurological treatments for patients manifesting muscular rigidity and loss of motor control in disease states such as Parkinson's disease. Unlike pharmaceutical therapeutics which are partially effective temporarily, transplantation of chimeric stem cells that regulate the production dopamine and serotonergic factors will offer these patients superior outcomes.

The invention has been described in terms of preferred embodiments thereof, but is more broadly applicable as will be understood by those skilled in the art. the scope of the invention is therefore limited only by the following claims.

What is claimed is:

1. A method of isolating and propagating a line of embryonic stem cells that originates from either morulae (pre-stem) or blastocyst (ICM stem cells)

2. The method of claim 1, wherein the propagated line of embryonic stem cells are used for the purpose of transplanting cells, tissues or organs.

3. The method of claim 1, wherein at least one regulatory factor is used to propagate the line of embryonic stem cells.

4. The method of claim 3, wherein the regulatory factor is derived from teacher cells or teacher cells' spent culture medium.

5. The method of claim 3, wherein the propagation is initiated by the formation of chimeric inner cell mass cells.

6. The method of claim 5, wherein the formation of chimeric inner cell mass cells comprises nuclear transplantation, mitochondrial substitution, or cytoplasmic depletion.

7. The method of claim 1, wherein the embryonic stem cells are cultured in a medium in the presence of at least one agent or cytokine in order to differentiate into specific cells or tissues.

8. The method of claim 7, wherein the agent or cytokine is selected from the group consisting of IL-1, TNF-α, IL-6, PTH, PDGF, PGE, cAMP, estrogens, anti-estrogens, progestins, anti-progestins, cortisol, GH, androgens, I₃/T₃, VGEF and cyclosporin.

9. The method of claim 7, wherein the concentration of the agent or cytokine in culture medium is from about 1.0 pg/ml to about 10.0 ng/ml.

10. The method of claim 7, wherein the specific cells are selected from the group consisting of nerve cells, bone cells, immune cells, and pancreatic beta cells.

11. The method of claim 7, wherein the embryonic stem cell differentiation is identified by at least one marker substance that accumulates in culture medium.

12. The method of claim 11, wherein the marker substance is selected from the group consisting of an iron sequestering substance, insulin, dopamine, myeloid fibers, and hemoglobin.

13. The method of claim 5, wherein the formation of chimeric inner cell mass cells enhances the proficiency of stem cells to both replicate and perform metabolic functions that restore essential body function.

14. The method of claim 1, wherein clonal properties of the propagated stem cells is achieved by adding apoptotic factors to the culture medium to eliminate contaminating members of the stem cells that did not properly differentiate.

15. The method of claim 1, wherein clonal properties of the propagated stem cells is achieved by adding at least one agent or cytokine to the culture medium to eliminate contaminating members of the stem cells that did not properly differentiate, wherein the agent or cytokine is selected from the group consisting of IL-1, TNF-α, IL-6, PTH, PDGF, PGE₂, cAMP, estrogens, anti-estrogens, progestins, anti-progestins, cortisol, GH, androgens, I₃/T₃, VGEF and cyclosporin.

16. The method of claim 1, wherein the propagation of the line of embryonic stem cells is done in vivo by transplanting teacher cells into an area sufficiently close to the embryonic stem cells to allow for at least one regulatory factor made by the teacher cells to contact the embryonic cells.

17. The method of claim 1, wherein the presence or absence of different concentrations of calcium is used to regulate the propagation of the line of embryonic stem cells.

18. The method of claim 1, wherein the propagated line of embryonic stem cells is grown in a three dimensional manner before being transplanted.
